(19) United States
(12) Patent Application Publication
(10) Pub. No.: US 2017/0065433 A1
(43) Pub. Date: Mar. 9, 2017

(54) SYSTEMS AND METHODS FOR MEASURING ORTHOPEDIC PARAMETERS IN ARTHROPLASTIC PROCEDURES

(71) Applicant: MiRus LLC, Atlanta, GA (US)

(72) Inventors: Angad Singh, Marietta, GA (US); Philip Matthew Fitzsimons, Lilburn, GA (US)

(21) Appl. No.: 15/266,044

(22) Filed: Sep. 15, 2016

Related U.S. Application Data
(63) Continuation of application No. 14/440,292, filed on May 1, 2015.
(60) Provisional application No. 61/722,102, filed on Nov. 2, 2012.

(51) Int. Cl. A61F 2/46; A61F 2/36; A61B 5/103; A61F 2/34; A61B 5/00; A61B 5/11

(52) U.S. Cl. CPC A61F 2/4657; A61B 5/4851; A61B 5/1122; A61B 5/103; A61F 2/34; A61F 2/3609; A61F 2/4684; A61F 2002/3067

(57) ABSTRACT

A force sensing module for measuring performance parameters associated with an orthopedic articular joint is disclosed. The force sensing module includes a housing having a substantially concave articular surface and an implant surface, the substantially concave articular surface and the implant surface defining a compartment therebetween. The force sensing module also includes a first set of sensors disposed within the compartment, which are mechanically coupled between the substantially concave articular surface and the implant surface. The first set of sensors is configured to detect information indicative of a first portion of a force present at a first area of the substantially concave articular surface. The force sensing module also includes a second set of sensors disposed within the compartment, which are mechanically coupled between the substantially concave articular surface and the implant surface. The second set of sensors is configured to detect information indicative of a second portion of the force present at a second area of the substantially concave articular surface.

### CROSS-REFERENCE TO RELATED APPLICATION

This application is a continuation of U.S. application Ser. No. 14/440,292, filed May 1, 2015, which is a 371 of PCT/US2013/068078, filed Nov. 1, 2013, which claims the benefit of U.S. Provisional Application No. 61/722,102, filed Nov. 2, 2012, the disclosures of which are incorporated herein by reference in their entireties.

### TECHNICAL FIELD

The present disclosure relates generally to orthopedic surgery and, more particularly, to systems and methods for measuring orthopedic parameters associated with a reconstructed joint in orthopedic arthroplastic procedures.

### BACKGROUND

For most surgical procedures, it is advantageous for a surgeon to compare intra-operative progress and post-operative results to ensure that surgical objectives are met. In some surgical procedures, particularly those involving orthopedic arthroplasty, relatively small procedural deviations can translate into significant differences in the functionality of the patient's anatomy. For example, in joint replacement surgery on the knee or hip, small deviations in the positioning of the prosthetic joint components or ligament imbalances may result in considerable differences in the patient's posture, gait, and/or range of motion.

During orthopedic procedures involving resurfacing, replacement, or reconstruction of ball-and-socket joints, such as in the hip, surgeons attempt to ascertain performance of a newly-implanted joint. The surgeon may evaluate the biomechanical stability of the joint and determine whether additional adjustment of the implant is required before finishing the surgery. One important aspect of joint performance for ball-and-socket joints, such as the hip or shoulder, is the magnitude and relative location of the forces as the joint is articulated through various poses and ranges of motion. For example, for a hip replacement procedure, the magnitude and location of forces applied by the femoral head on the hip socket (or the acetabular cup in a reconstructed joint) provide a strong indication of the stability of the joint; larger forces at the perimeter of the socket tend to increase the possibility of a dislocation, subluxation, or femoral impingement.

Currently intra-operative evaluation of the stability of a reconstructed joint is highly subjective. The evaluation process typically involves the surgeon manually placing the leg in different poses and repeatedly articulating the joint through varying degrees of joint angles such as flexion and extension while testing the range of motion and relative stability of the joint based on “look and feel.” This process for intra-operative evaluation is extremely subjective, and the performance of the reconstructed joint is highly dependent on the experience level of the surgeon. Perhaps not surprisingly, it is difficult for patients and doctors to reliably predict the relative success of the surgery (and the need for subsequent corrective/adjustment surgeries) until well after the initial procedure. Such uncertainty has a negative impact on long term clinical outcomes, patient quality of life, and the ability to predict and control costs associated with surgery, recovery, and rehabilitation.

In order to limit or remove the uncertainty and imprecision associated with the “look and feel” approaches in intra-operative joint evaluation, it would be advantageous for surgeons to be able to evaluate, in real-time or near real-time, certain objective orthopedic performance parameters. For example solutions that measure kinematic and kinetic parameters simultaneously would be of particular interest.

The presently disclosed systems and methods for intra-operatively measuring performance parameters in orthopedic arthroplastic procedures are directed to overcoming one or more of the problems set forth above and/or other problems in the art.

### SUMMARY

According to one aspect, the present disclosure is directed to a force sensing module for measuring performance parameters associated with an orthopedic articular joint. The force sensing module may comprise a housing including a substantially concave articular surface and an implant surface, the substantially concave articular surface and the implant surface defining a compartment therebetween. The force sensing module may also comprise a first set of sensors disposed within the compartment, the first set of sensors being mechanically coupled between the substantially concave articular surface and the implant surface. The first set of sensors may be configured to detect information indicative of a first portion of a force present at a first area of the substantially concave articular surface. The force sensing module may further comprise a second set of sensors disposed within the compartment, the second set of sensors being mechanically coupled between the substantially concave articular surface and the implant surface. The second set of sensors may be configured to detect information indicative of a second portion of the force present at a second area of the substantially concave articular surface. Additional sets of sensors may be configured to detect forces in other areas.

In accordance with another aspect, the present disclosure is directed to a force sensing module for measuring performance parameters associated with an orthopedic articular joint. The force sensing module may comprise a housing having a substantially convex articular surface defining a compartment therewithin. The force sensing module may also comprise a plurality of sensors disposed within the compartment, each sensor being mechanically coupled to the substantially convex articular surface and configured to detect information indicative of a respective portion of a force present at the substantially concave articular surface.

In accordance with another aspect, the present disclosure is directed to a joint angle measuring system consisting of at least one inertial measurement unit to measure the angle of an orthopedic articular joint. The orientation sensing system may comprise at least one inertial measurement unit configured to detect information indicative of a 3-dimensional orientation of the moving bone or bones in a joint. The inertial measurement unit(s) may be embedded in the joint prosthesis or rigidly attached to a part or parts of the patient's anatomy. Alternatively, the inertial measurement unit may be integrated with the force sensing module described above.

According to another aspect, the present disclosure is directed to a computer-implemented method for tracking performance parameters associated with an orthopedic articular joint, the method comprising receiving, at a processor associated with a computer, first information indicative of a force detected at an articular surface of an acetabular prosthetic component of a patient. The method may also comprise estimating, by the processor, a location of a center of the force relative to the articular surface of the acetabular prosthetic component, the estimated location based, at least in part, on the first information. The method may further comprise providing, by the processor, second information indicative of the estimated location of the center of the force relative to the approximate center of the articular surface of the acetabular prosthetic component. The method may further comprise receiving, at a processor associated with a computer, third information indicative of 3-dimensional orientation of the moving bone or bones that comprise the joint and estimating, by the processor, a fourth information indicative of the 3-dimensional joint angles, the estimated joint angle based, at least in part, on the third information.

In accordance with another aspect, the present disclosure is directed to a force sensing trial implant system for intra-operatively measuring performance parameters associated with an orthopedic articular joint. The force sensing trial implant system comprises a first component having a first housing that includes a substantially concave articular surface and an implant surface, the substantially concave articular surface and the implant surface defining a first compartment therebetween. The first component may comprise at least one metallic material disposed within the first compartment at predetermined distance from the substantially concave articular surface. The force sensing trial implant system may comprise a second component having a second housing that includes a substantially convex articular surface defining a second compartment therewithin. The second component may comprise at least one coil component disposed within the second compartment at a predetermined position relative to the substantially convex articular surface, and a processor disposed within the second compartment and coupled to at least one coil component. The articular surface of the first component is configured to compress in response to a force applied by the substantially convex articular surface of the second component, such that the compression of the substantially concave articular surface results in a change in the proximity of at least one coil of the second component with at least one metallic material of the first component. The compression or deflection characteristics of the articular surface are known to the extent necessary to relate the compressed distance to the applied force. The processor may be configured to measure an inductance value associated with the at least one coil which is proportional to the distance between the coil and the metallic material.

### BRIEF DESCRIPTION OF THE DRAWINGS

FIG. 1 provides a front view of a portion of an exemplary hip joint, the type of which may be involved in a joint replacement procedure consistent with certain disclosed embodiments;

FIG. 2A provides a schematic view of exemplary components associated with a prosthetic hip joint, which may be used in a joint replacement procedure consistent with the disclosed embodiments;

FIG. 2B illustrates a magnified view of an exemplary prosthetic hip joint in a reduced state in accordance with certain disclosed embodiments;

FIG. 3 provides a perspective view of an exemplary hip prosthetic system in a fully reduced (reconstructed) state, which may be used in a total hip arthroplastic (THA) procedure, consistent with certain disclosed embodiments;

FIG. 4 provides a diagrammatic view of an exemplary orthopedic performance monitoring system (embodied as an intra-operative total hip arthroplasty (THA) performance monitoring system) consistent with certain disclosed embodiments;

FIG. 5 provides a schematic view of exemplary components associated with an orthopedic performance monitoring system, such as the THA performance monitoring system illustrated in FIG. 3;

FIG. 6A provides a perspective exploded view of an exemplary trial prosthetic hip implant system including a force sensing module consistent with certain disclosed embodiments;

FIG. 6B provides a perspective exploded view of an alternative exemplary trial prosthetic hip implant system including a force sensing module, in accordance with certain disclosed embodiments;

FIG. 6C provides a perspective exploded view of yet another exemplary trial prosthetic hip implant system including a force sensing module consistent with certain disclosed embodiments;

FIG. 7A provides a cross-sectional side view of implantable acetabular cup trial prosthetic components including a force sensing module, consistent with certain disclosed embodiments;

FIG. 7B provides a schematic top view of implantable acetabular cup trial prosthetic components illustrating an exemplary arrangement of certain elements of force sensing module, consistent with certain disclosed embodiments;

FIG. 8 provides a schematic internal view of a force sensing trial implant system, in accordance with certain disclosed embodiments;

FIG. 9 illustrates an embodiment of a user interface that may be provided on a monitor or output device for intra-operatively displaying the monitored joint performance parameters in real time consistent with certain disclosed embodiments;

FIG. 10 illustrates another embodiment of a user interface that may be provided on a monitor or output device for intra-operatively displaying the monitored joint performance parameters in real time, in accordance with the disclosed embodiments; and

FIG. 11 provides a flowchart depicting an exemplary process to be performed by one or more processing devices associated with orthopedic performance monitoring systems, consistent with certain disclosed embodiments.

### DETAILED DESCRIPTION

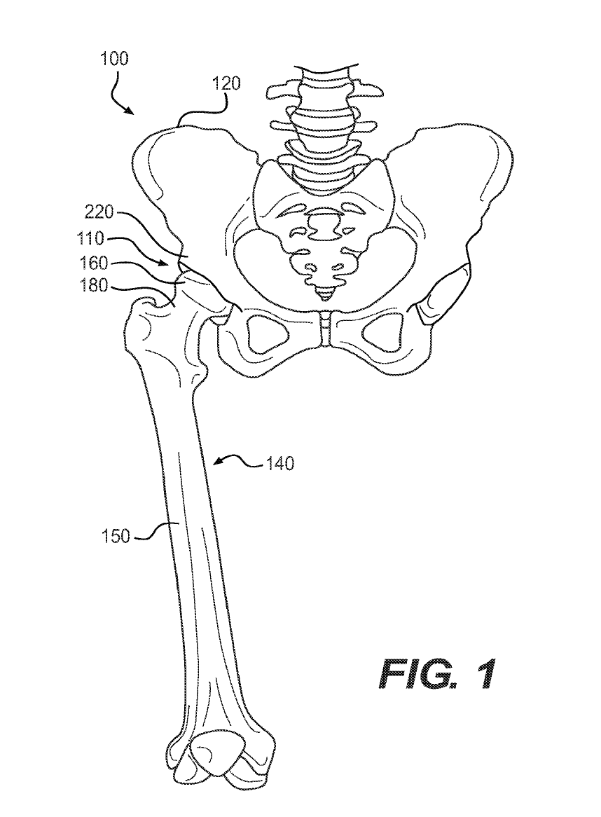

FIG. 1 illustrates a front view of an exemplary portion of the pelvic region 100 of the human body, which includes a hip joint 110. Proper articulation of hip joint 110 contributes to many basic structural and motor functions of the human body, such as standing and walking. As illustrated in FIG. 1, hip joint 110 comprises the interface between pelvis 120 and the proximal end of femur 140. The proximal end of femur 140 includes a femoral head 160 disposed on a femoral neck 180. Femoral neck 180 connects femoral head 160 to a femoral shaft 150. Femoral head 160 fits into a concave socket in pelvis 120 called the acetabulum 220. Acetabulum 220 and femoral head 160 are both covered by articular cartilage (not shown) that absorbs shock and promotes articulation of hip joint 110.

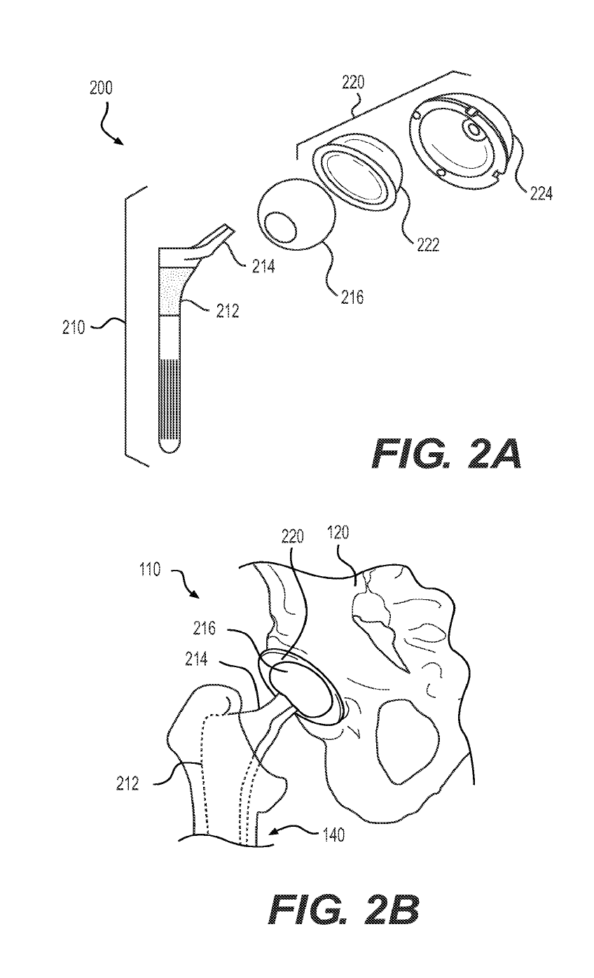

Over time, hip joint 110 may degenerate (due, for example, to osteoarthritis) resulting in pain and diminished functionality of the joint. As a result, a hip replacement procedure, such as total hip arthroplasty or hip resurfacing, may be necessary. During a hip replacement procedure, a surgeon may replace portions of hip joint 110 with artificial prosthetic components. For example, in one type of hip replacement procedure—called total hip arthroplasty (THA)—the surgeon may remove femoral head 160 and neck 180 from femur 140 and replace them with a femoral prosthesis. Similarly, the surgeon may resect or resurface portions of acetabulum 220 using a surgical reamer or reciprocating saw, and may replace the removed portions of acetabulum 220 with a prosthetic acetabular cup. Prosthetic components associated with the hip joint 110 are illustrated in FIG. 2A.

As illustrated in FIG. 2A, the natural (or “native”) femoral components removed during the arthroplasty may be replaced with a prosthetic femoral component 200 comprising a prosthetic head 216, a prosthetic neck 214, and a stem 212. Stem 212 of prosthetic femoral component 26 is typically anchored in a cavity that the surgeon creates in the intramedullary canal of femur 140.

Similarly, the native acetabular components removed during the hip replacement procedure may be replaced with a prosthetic acetabular component 220 comprising a cup 224 that may include a liner 222. To install acetabular component 220, the surgeon connects cup 224 to a distal end of an impactor tool and implants cup 224 into the reamed acetabulum 220 by repeatedly applying force to a proximal end of the impactor tool. If acetabular component 220 includes a liner 222, the surgeon snaps liner 222 into cup 224 after implanting cup 224 within acetabulum 220.

FIG. 2B illustrates a magnified view of an exemplary prosthetic hip joint in a reduced (i.e., assembled) state. As illustrated in FIG. 2B, the stem 212 is secured within the intramedullary canal of femur 140. The prosthetic head 216 is engaged with the acetabular component 220 of pelvis 120 to form the new prosthetic joint. Before completing the surgery, the surgeon may compare certain functional parameters of the reduced prosthetic joint to determine whether the prosthetic joint is positioned properly, is biomechanically stable, and provides the joint with adequate range of motion. Methods and systems consistent with the disclosed embodiments provide a solution for measuring performance parameters (e.g., magnitude and location of force relative to the patient's reconstructed orthopedic anatomy) during intra-operative evaluation of the reduced joint. Such methods and systems will be described in greater detail below.

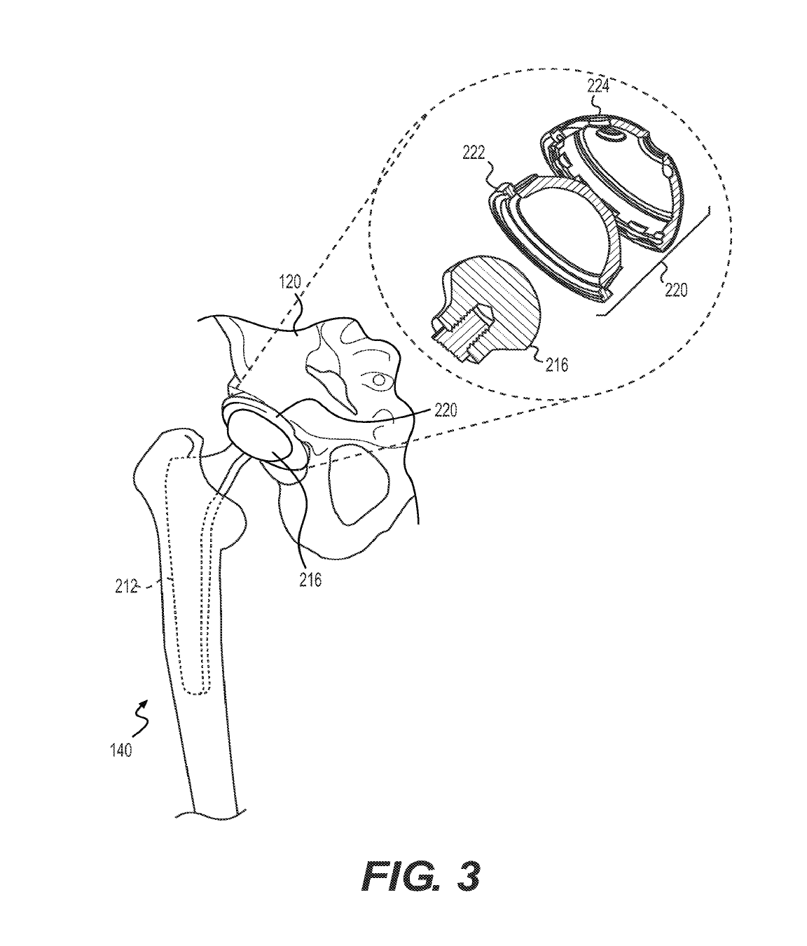

FIG. 3 provides a magnified view of hip joint 110 showing a modular trial prosthetic hip system in which the presently disclosed force sensing and/or joint angle measurement components of orthopedic performance monitoring system 300 may be implemented. According to one embodiment, a performance monitoring package (e.g., a force sensing module 230 and an inertial measurement unit 221, both of which are described in further detail below) may be embedded within the head 216 of a femoral trial prosthetic component. Alternatively or additionally, the performance monitoring package (or a portion thereof), may be embedded within one or more of the acetabular trial prosthetic components 220. According to yet another embodiment, components of the performance monitoring package may be distributed across different components. For example, an inertial measurement unit 221 may be installed or embedded within head 216 of femoral trial prosthetic component, while the force monitoring module 230 may be installed or embedded within one or more of the acetabular trial prosthetic components 220. Regardless of which configuration is used, the presently disclosed systems for intra-operatively measuring performance parameters in orthopedic arthroplastic procedures are designed to replicate the shape, size, and performance of the femoro-pelvic interface of a reconstructed, fully-reduce reduced joint, thereby ensuring more accurate performance measurement results and more reliable prediction of post-operative joint performance.

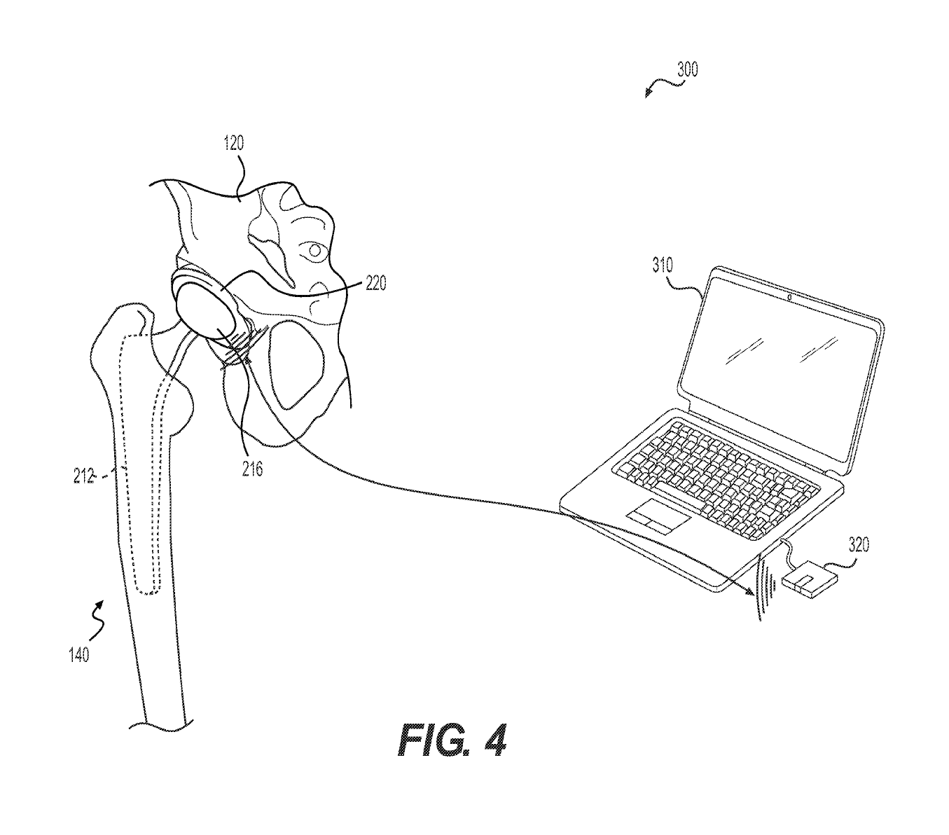

FIG. 4 provides a diagrammatic illustration of an exemplary orthopedic performance monitoring system 300 for intra-operative detection, monitoring, and tracking of forces present at an orthopedic joint, such as hip joint 110. Those skilled in the art will recognize that embodiments consistent with the presently disclosed systems and methods may be employed in any environment involving arthroplastic procedures related to ball-and-socket joints, such as the hip and shoulder. Furthermore, certain embodiments consistent with the presently disclosed systems and methods may be used in non-surgical applications to, for example, measure and track the force loading profile of almost any ball-and-socket joint.

For example, in accordance with the exemplary embodiment illustrated in FIG. 4, orthopedic performance monitoring system 300 may embody a system for intra-operatively—and in real-time or near real-time—gathering, analyzing, and tracking anatomical performance parameters at hip joint 110 during a full or partial hip replacement procedure. Performance parameters may include or embody any kinetic, kinematic or biomechanical parameter that may be used to characterize the behavior or performance of an orthopedic joint. Non-limiting examples of performance parameters include any information indicative of force, pressure, angle of flexion and/or extension, torque, angle of abduction and/or adduction, location of center of force, angle of internal/external rotation, range of motion, or orientation. Orthopedic performance monitoring system 300 may be configured to monitor one or more of these exemplary performance parameters, track the performance parameter(s) over time, and display the monitored and/or tracked data to a surgeon or medical professional in real-time. As such, orthopedic performance monitoring system 300 provides a platform that facilitates real-time intra-operative evaluation of several joint performance parameters simultaneously. Individual components of exemplary embodiments of orthopedic performance monitoring system 300 will now be described in more detail.

FIG. 4 illustrates a schematic diagram of orthopedic performance monitoring system 300. As illustrated in FIG. 4, orthopedic performance monitoring system 300 may include a force sensing module 230, one or more inertial measurement units 221, a processing device (such as processing system 310 (or other computer device for processing data received by orthopedic performance monitoring system 300)), and one or more wireless communication transceivers 320 for communicating with one or more of force sensing module 230 or one or more inertial measurement units 221. The components of orthopedic performance monitoring system 300 described above are exemplary only, and are not intended to be limiting. Indeed, it is contemplated that additional and/or different components may be included as part of orthopedic performance monitoring system 300 without departing from the scope of the present disclosure. For example, although wireless communication transceiver 320 is illustrated as being a standalone device, it may be integrated within one or more other components, such as processing system 310. Thus, the configuration and arrangement of components of orthopedic performance monitoring system 300 illustrated in FIG. 4 are intended to be exemplary only.

Processing system 310 may include or embody any suitable microprocessor-based device configured to process and/or analyze information indicative of performance of an articular joint. According to one embodiment, processing system 310 may be a general purpose computer programmed with software for receiving, processing, and displaying information indicative of performance parameters associated with the articular joint. According to other embodiments, processing system 310 may be a special-purpose computer, specifically designed to communicate with, and process information for, other components associated with orthopedic performance monitoring system 300. Individual components of, and processes/methods performed by, processing system 310 will be discussed in more detail below.

Processing system 310 may be communicatively coupled to one or more of force sensing module 230 and inertial measurement unit 221 and may be configured to receive, process, and/or analyze data monitored by force sensing module 230 and/or inertial measurement unit 221. According to one embodiment, processing system 310 may be wirelessly coupled to each of force sensing module 230 and inertial measurement unit 221 via wireless communication transceiver(s) 320 operating any suitable protocol for supporting wireless (e.g., wireless USB, ZigBee, Bluetooth, Wi-Fi, etc.) In accordance with another embodiment, processing system 310 may be wirelessly coupled to one of force sensing module 230 or inertial measurement unit 221, which, in turn, may be configured to collect data from the other constituent sensors and deliver it to processing system 310.

Wireless communication transceiver(s) 320 may include any device suitable for supporting wireless communication between one or more components of orthopedic performance monitoring system 300. As explained above, wireless communication transceiver(s) 320 may be configured for operation according to any number of suitable protocols for supporting wireless, such as, for example, wireless USB, ZigBee, Bluetooth, Wi-Fi, or any other suitable wireless communication protocol or standard. According to one embodiment, wireless communication transceiver 320 may embody a standalone communication module, separate from processing system 310. As such, wireless communication transceiver 320 may be electrically coupled to processing system 310 via USB or other data communication link and configured to deliver data received therein to processing system 310 for further processing/analysis. According to other embodiments, wireless communication transceiver 320 may embody an integrated wireless transceiver chipset, such as the Bluetooth, Wi-Fi, NFC, or 802.11x wireless chipset included as part of processing system 320.

Force sensing module 230 may include a plurality of components that are collectively adapted for implantation within at least a portion of an articular joint and configured to detect various forces and other performance parameters present at, on, and/or within the articular joint. According to one embodiment, force sensing module 230 may be included as part of a trial prosthetic component system that is configured for temporary implantation within a patient during, for example, a joint replacement procedure, such as a total or partial hip replacement or reconditioning procedure.

Force sensing module 230 may be configured to be embedded within a trial acetabular prosthetic component 220. For example, according to one embodiment, force sensing module 230 may be disposed within an acetabular cup 224 that is affixed to the pelvis 120 of a patient. In another embodiment, force sensing module 230 may be disposed within an acetabular liner 222 that is designed for near-frictionless articulation with a corresponding portion of femoral prosthetic head 216 component. In either embodiment, force sensing module 230 may be disposed within a housing compartment that is formed between a substantially concave articular surface (e.g., the surface that receives the head 216 of a femoral prosthetic implant 200 or the femoral head 160 of the patient) and the implant surface (e.g., the surface that interfaces with the patient pelvic bone 120).

Inertial measurement unit 221 may be any system suitable for measuring information that can be used to accurately measure orientation in 3 dimensions. From this orientation information the joint angles such as the angle or amount of flexion and/or extension, the angle or amount of abduction and/or adduction, or the internal/external rotation of the articular joint may be derived. According to one embodiment, at least one inertial measurement unit 221 is attached or embedded within a portion of the femoral prosthetic component 200. In another embodiment, at least one inertial measurement unit 221 is attached or embedded within a portion of the acetabular prosthetic component 220 and used in combination with the first embodiment above, in order to more precisely account for the positions of the patient's femur with respect to the pelvis. Alternatively or additionally, inertial measurement unit 221 may be attached to the patient's leg, or any other part of the patient's anatomy that is indicative of the movement of the femur relative to the pelvis. In some embodiments, two inertial measurement units 221 may be used—one of which is attached to the patient's femur and the other of which is attached to the patient's pelvis, in order to more precisely account for the positions of the patient's femur with respect to the pelvis.

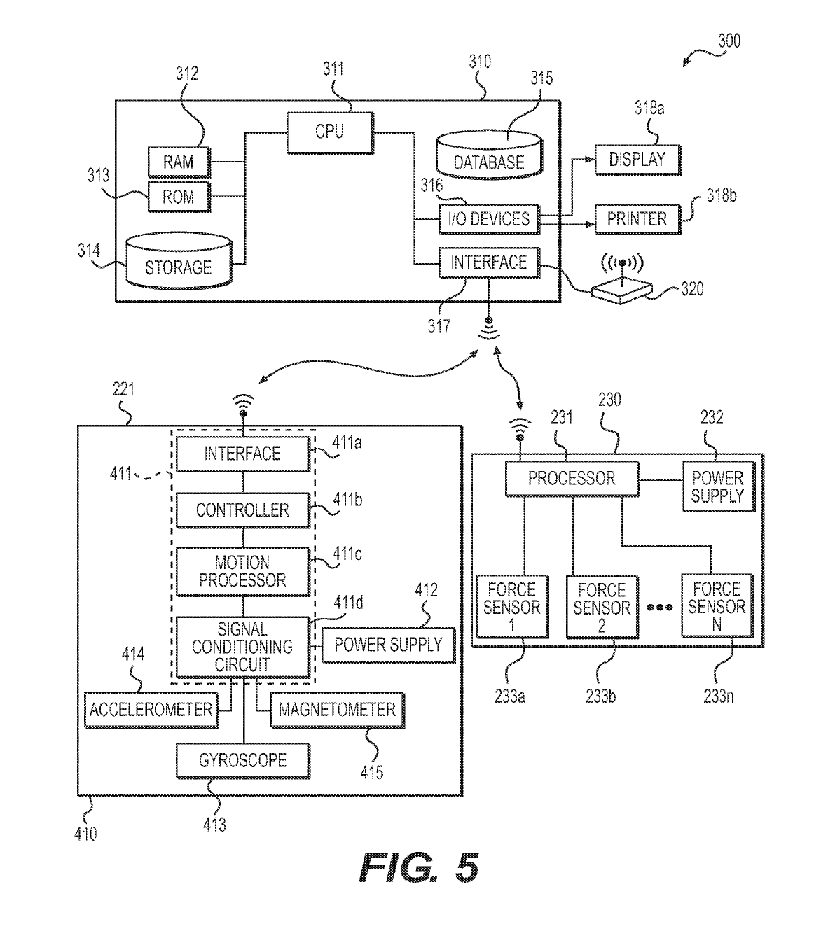

FIG. 5 provides a schematic diagram illustrating certain exemplary subsystems associated with orthopedic performance monitoring system 300 and its constituent components. Specifically, FIG. 3 is a schematic block diagram depicting exemplary subcomponents of processing system 310, force sensing module 230, and one or more inertial measurement units 221 in accordance with certain disclosed embodiments.

As explained, processing system 310 may be any processor-based computing system that is configured to receive performance parameters associated with an orthopedic joint 110, analyze the received performance parameters to extract data indicative of the performance of orthopedic joint 110, and output the extracted data in real-time or near real-time. Non-limiting examples of processing system 310 include a desktop or notebook computer, a tablet device, a smartphone, wearable or handheld computers, or any other suitable processor-based computing system.

For example, as illustrated in FIG. 5, processing system 310 may include one or more hardware and/or software components configured to execute software programs, such as software tracking performance parameters associated with orthopedic joint 110 and displaying information indicative of the performance of the joint. According to one embodiment, processing system 310 may include one or more hardware components such as, for example, a central processing unit (CPU) or microprocessor 311, a random access memory (RAM) module 312, a read-only memory (ROM) module 313, a memory or data storage module 314, a database 315, one or more input/output (I/O) devices 316, and an interface 317. Alternatively and/or additionally, processing system 310 may include one or more software media components such as, for example, a computer-readable medium including computer-executable instructions for performing methods consistent with certain disclosed embodiments. It is contemplated that one or more of the hardware components listed above may be implemented using software. For example, storage 314 may include a software partition associated with one or more other hardware components of processing system 310. Processing system 310 may include additional, fewer, and/or different components than those listed above. It is understood that the components listed above are exemplary only and not intended to be limiting.

CPU 311 may include one or more processors, each configured to execute instructions and process data to perform one or more functions associated with processing system 310. As illustrated in FIG. 5, CPU 311 may be communicatively coupled to RAM 312, ROM 313, storage 314, database 315, I/O devices 316, and interface 317. CPU 311 may be configured to execute sequences of computer program instructions to perform various processes, which will be described in detail below. The computer program instructions may be loaded into RAM 312 for execution by CPU 311.

RAM 312 and ROM 313 may each include one or more devices for storing information associated with an operation of processing system 310 and/or CPU 311. For example, ROM 313 may include a memory device configured to access and store information associated with processing system 310, including information for identifying, initializing, and monitoring the operation of one or more components and subsystems of processing system 310. RAM 312 may include a memory device for storing data associated with one or more operations of CPU 311. For example, ROM 313 may load instructions into RAM 312 for execution by CPU 311.

Storage 314 may include any type of mass storage device configured to store information that CPU 311 may need to perform processes consistent with the disclosed embodiments. For example, storage 314 may include one or more magnetic and/or optical disk devices, such as hard drives, CD-ROMs, DVD-ROMs, or any other type of mass media device. Alternatively or additionally, storage 314 may include flash memory mass media storage or other semiconductor-based storage medium.

Database 315 may include one or more software and/or hardware components that cooperate to store, organize, sort, filter, and/or arrange data used by processing system 310 and/or CPU 311. For example, database 315 may include historical data such as, for example, stored performance data associated with the orthopedic joint. CPU 311 may access the information stored in database 315 to provide a performance comparison between previous joint performance and current (i.e., real-time) performance data. CPU 311 may also analyze current and previous performance parameters to identify trends in historical data (i.e., the forces detected at various joint angles with different prosthesis designs and patient demographics). These trends may then be recorded and analyzed to allow the surgeon or other medical professional to compare the force data at various joint angles with different prosthesis designs and patient demographics. It is contemplated that database 315 may store additional and/or different information than that listed above.

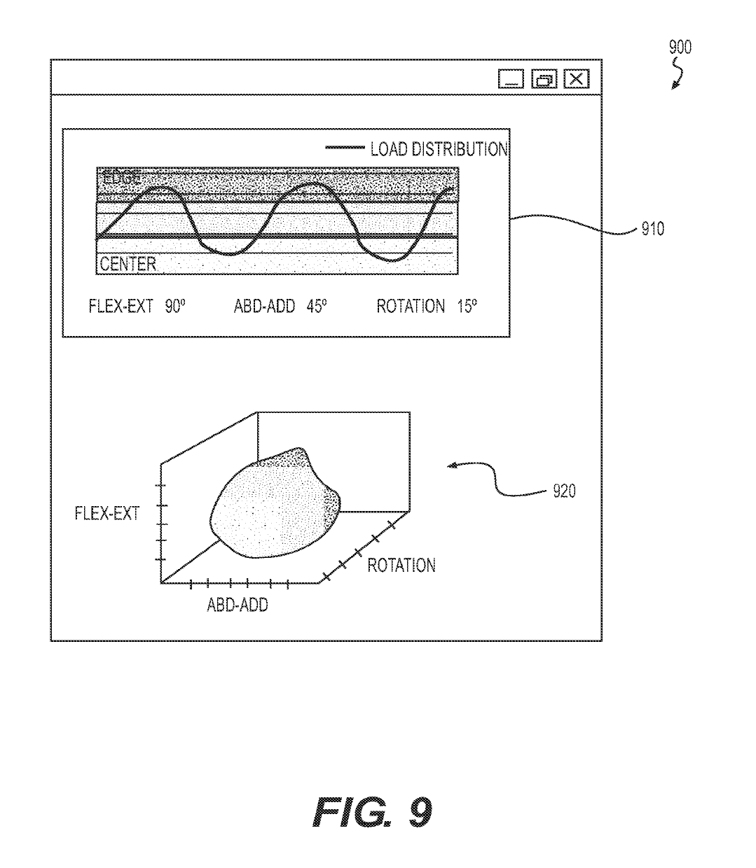

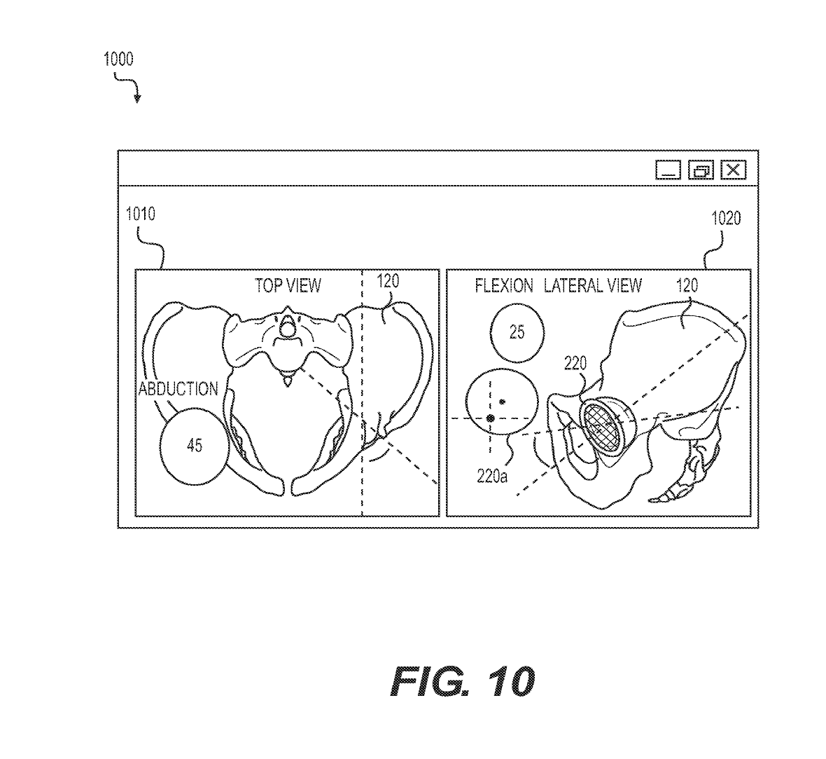

I/O devices 316 may include one or more components configured to communicate information with a user associated with orthopedic performance monitoring system 300. For example, I/O devices may include a console with an integrated keyboard and mouse to allow a user to input parameters associated with processing system 310. I/O devices 316 may also include a display including a graphical user interface (GUI) (such as GUI 900 shown in FIG. 9 or GUI 1000 shown in FIG. 10) for outputting information on a display monitor 318a. I/O devices 316 may also include peripheral devices such as, for example, a printer 318b for printing information associated with processing system 310, a user-accessible disk drive (e.g., a USB port, a floppy, CD-ROM, or DVD-ROM drive, etc.) to allow a user to input data stored on a portable media device, a microphone, a speaker system, or any other suitable type of interface device.

Interface 317 may include one or more components configured to transmit and receive data via a communication network, such as the Internet, a local area network, a workstation peer-to-peer network, a direct link network, a wireless network, or any other suitable communication platform. For example, interface 317 may include one or more modulators, demodulators, multiplexers, demultiplexers, network communication devices, wireless devices, antennas, modems, and any other type of device configured to enable data communication via a communication network. According to one embodiment, interface 317 may be coupled to or include wireless communication devices, such as a module or modules configured to transmit information wirelessly using Wi-Fi or Bluetooth wireless protocols. Alternatively or additionally, interface 317 may be configured for coupling to one or more peripheral communication devices, such as wireless communication transceiver 320.

As explained, inertial measurement unit(s) 221 may include one or more subcomponents configured to detect and transmit information that either represents 3-dimensional orientation or can be used to derive an orientation of the inertial measurement unit 221 (and, by extension, any object that affixed relative to inertial measurement unit 221, such as a femur or pelvis of a patient). Inertial measurement unit(s) 221 may embody a device capable of determining a 3-dimensional orientation associated with any body to which inertial measurement unit(s) 221 is/are attached. According to one embodiment, inertial measurement unit(s) 221 may include a microprocessor 411, a power supply 412, and one or more of a gyroscope 413, an accelerometer 414, or a magnetometer 415.

According to one embodiment, inertial measurement unit(s) 221 may contain a 3-axis gyroscope 413, a 3-axis accelerometer 414, and a 3-axes magnetometer 415. It is contemplated, however, that fewer of these devices with fewer axes can be used without departing from the scope of the present disclosure. For example, according to one embodiment, inertial measurement units may include only a gyroscope and an accelerometer, the gyroscope for calculating the orientation based on the rate of rotation of the device, and the accelerometer for measuring earth's gravity and linear motion, the accelerometer providing corrections to the rate of rotation information (based on errors introduced into the gyroscope because of device movements that are not rotational or errors due to biases and drifts). In other words, the accelerometer may be used to correct the orientation information collecting by the gyroscope. Similar the magnetometer 245 can be utilized to measure the earth's magnetic field and can be utilized to further correct gyroscope errors. Thus, while all three of gyroscope 243, accelerometer 244, and magnetometer 245 may be used, orientation measurements may be obtained using as few as one of these devices. The use of additional devices increases the resolution and accuracy of the orientation information and, therefore, may be advantageous when orientation accuracy is important.

As illustrated in FIG. 5, microprocessor 411 of inertial measurement unit 221 may include different processing modules or cores, which may cooperate to perform various processing functions. For example, microprocessor 411 may include, among other things, an interface 411a, a controller 411b, a motion processor 411c, and signal conditioning circuitry 411d. Controller 411b may be configured to control and receive conditioned and processed data from one or more of gyroscope 413, accelerometer 414, and magnetometer 415 and transmit the received data to one or more remote receivers. The data may be pre-conditioned via signal conditioning circuitry 411d, which includes amplifiers and analog-to-digital converters or any such circuits. The signals may be further processed by a motion processor 411c. Motion processor 411c may be programmed with so-called “motion fusion” algorithms to collect and process data from different sensors to generate error corrected orientation information. Accordingly, controller 411b may be communicatively coupled (e.g., wirelessly via interface 411a as shown in FIG. 3, or using a wireline protocol) to, for example, processing system 150 and configured to transmit the orientation data received from one or more of gyroscope 413, accelerometer 414, and magnetometer 415 to processing system 150, for further analysis.

Interface 411a may include one or more components configured to transmit and receive data via a communication network, such as the Internet, a local area network, a workstation peer-to-peer network, a direct link network, a wireless network, or any other suitable communication platform. For example, interface 411a may include one or more modulators, demodulators, multiplexers, demultiplexers, network communication devices, wireless devices, antennas, modems, and any other type of device configured to enable data communication via a communication network. According to one embodiment, interface 411a may be coupled to or include wireless communication devices, such as a module or modules configured to transmit information wirelessly using Wi-Fi or Bluetooth wireless protocols. As illustrated in FIG. 5, inertial measurement unit(s) 221 may be powered by power supply 412, such as a battery, fuel cell, MEMs micro-generator, or any other suitable compact power supply.

Importantly, although microprocessor 411 of inertial measurement unit 221 is illustrated as containing a number of discreet modules, it is contemplated that such a configuration should not be construed as limiting. Indeed, microprocessor 411 may include additional, fewer, and/or different modules than those described above with respect to FIG. 5, without departing from the scope of the present disclosure. Furthermore, in other instances of the present disclosure that describe a microprocessor (e.g., microprocessor 231 of force sensing module 230) are contemplated as being capable of performing many of the same functions as microprocessor 411 of inertial measurement unit 221 (e.g., signal conditioning, wireless communications, etc.) even though such processes are not explicitly described with respect to microprocessor 231. Those skilled in the art will recognize that many microprocessors include additional functionality (e.g., digital signal processing functions, data encryption functions, etc.) that are not explicitly described here. Such lack of explicit disclosure should not be construed as limiting. To the contrary, it will be readily apparent to those skilled in the art that such functionality is inherent to processing functions of many modern microprocessors, including the ones described herein.

Microprocessor 411 may be configured to receive data from one or more of gyroscope 413, accelerometer 414, and magnetometer 415 and transmit the received data to one or more remote receivers. Accordingly, microprocessor 411 may be communicatively coupled (e.g., wirelessly (as shown in FIG. 5, or using a wireline protocol) to, for example, processing system 310 and configured to transmit the orientation data received from one or more of gyroscope 413, accelerometer 414, and magnetometer 415 to processing system 310, for further analysis. As illustrated in FIG. 3, microprocessor 221 may be powered by power supply 412, such as a battery, fuel cell, MEMs micro-generator, or any other suitable compact power supply.

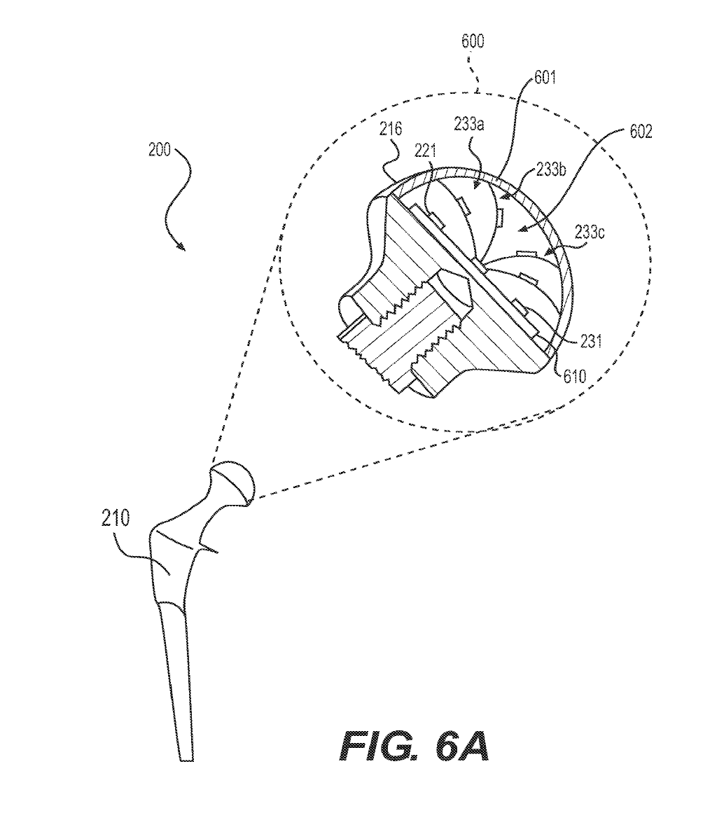

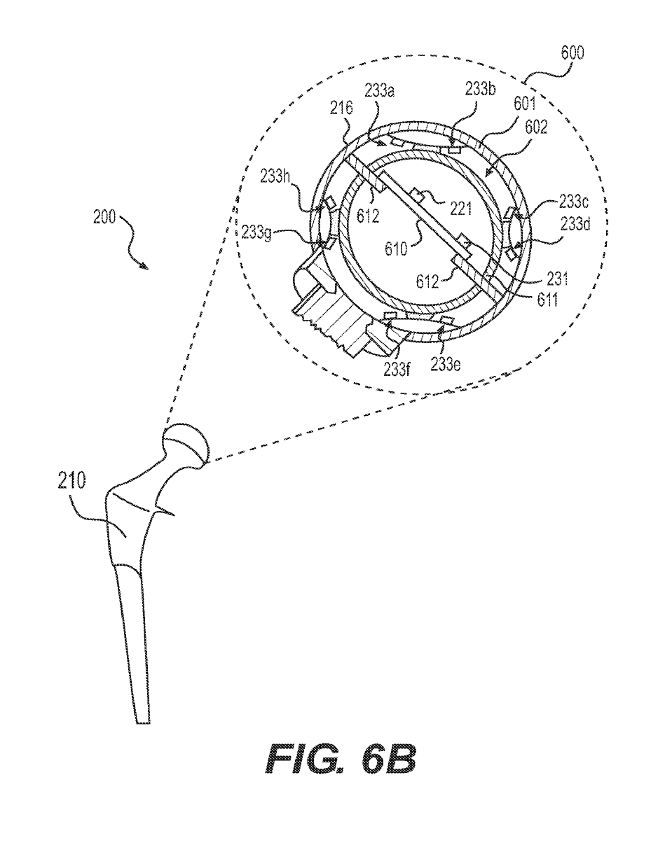

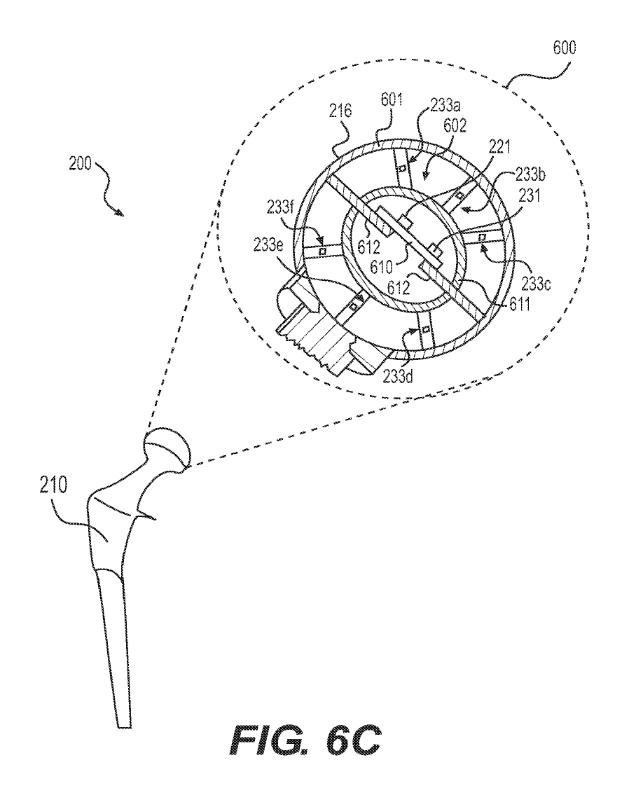

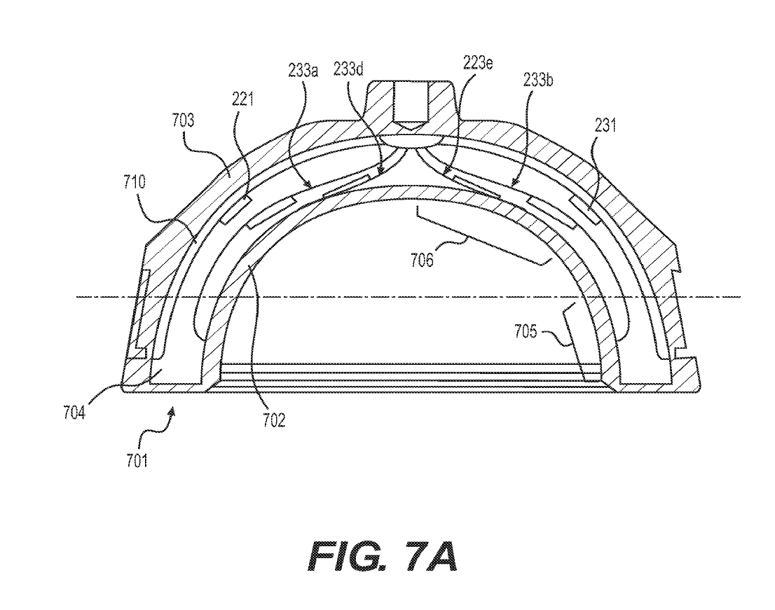

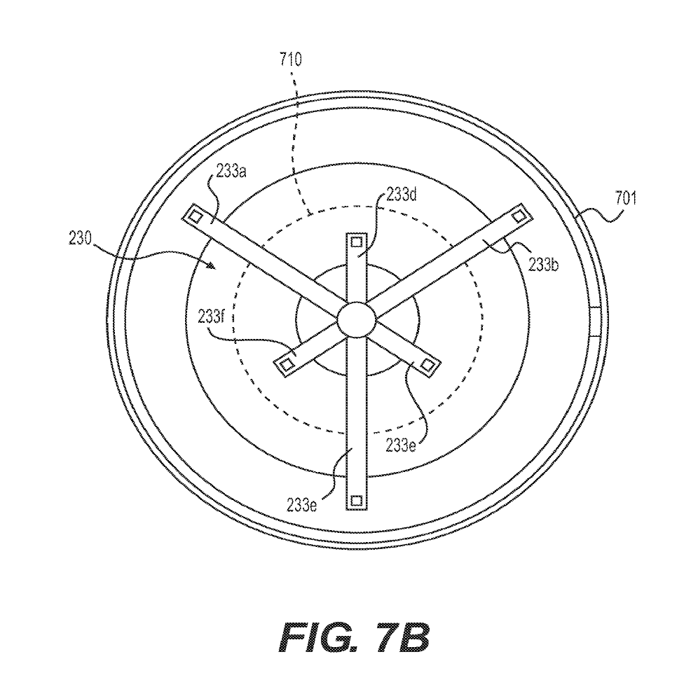

Force sensing module 230 may include a plurality of subcomponents that cooperate to detect force and performance data and, in certain embodiments, joint and/or femoral or pelvic component orientation information at orthopedic joint 110, and transmit the detected data to processing system 310, for further analysis. According to one exemplary embodiment, force sensing module 230 may include a microprocessor 231, a power supply 232, and one or more force sensors 233a, 233b, . . . , 233n. Those skilled in the art will recognize that the listing of components of force sensing module 230 is exemplary only and not intended to be limiting. Indeed, it is contemplated that force sensing module 230 may include additional and/or different components than those shown in FIG. 3, such as, for example, one or more integrated inertial measurement units (e.g., motion sensors, orientation sensors, etc.) Exemplary subcomponents of force sensing module 230 will be described in greater detail below with respect to FIGS. 6A-6C and 7A-7B. It is also contemplated that microprocessor 231 of force sensing module 230 may contain additional processing modules and/or subcomponents similar to those described, for example, in connection with microprocessor 411 of inertial measurement unit 221. Furthermore, although force sensing module 230 and inertial measurement unit 221 are illustrated as separate components, it is contemplated that, in certain embodiments, the force sensing and inertial measurement capabilities may be combined into a single system (and, in certain embodiments, within a single housing or as part of the same electronic circuit package). In such situations (such as when inertial measurement capabilities are combined with force sensing module 230, redundant modules (such as microprocessor) are not necessarily required.

FIG. 6A illustrates an exemplary perspective cross-sectional view of an embodiment in which force sensing module 230 is embedded as part of head 216 of trial hip prosthesis 200. As illustrated in FIG. 6A, head 216 may include a rigid or semi-rigid housing having a substantially convex articular surface 601. According to one embodiment, the substantially convex articular surface 601 of the housing may be substantially hemispheroidal in shape, the boundary of which defines a compartment 602 within the interior of head 216. The substantially convex articular surface 601 (as well as the housing as a whole) may be made of a material with known (or calibrated) structural integrity, such that forces acting on the substantially convex articular surface 601 are transferred to force sensors (such as force sensors 233a-233d) disposed within compartment 602.

In the embodiment shown in FIG. 6A, force sensing module 230 may include an electronic circuit board 610, a microprocessor 231 (and corresponding power supply (not shown)), a plurality of force sensors 233a-233d, and inertial measurement unit 221, each of which may be disposed within compartment 602. It is contemplated that additional and/or different components than those shown in FIG. 6A may be provided without departing from the scope of the present disclosure. For example, inertial measurement unit 221 may be excluded from head 216 altogether, or may be provided as an external component, affixed to some other portion of femoral prosthetic component 200.

Electronic circuit board 610 may include or embody any suitable material on which electronic circuits, such as processor 231, power supply (not shown), and inertial measurement unit 221 may be electrically coupled. For example, electronic circuit board 610 may embody a printed circuit board (PCB), multi-chip module (MCM), or flex circuit board. Electronic circuit board 610 may be configured to provide both integrated, space-efficient electronic packaging and mechanical support for the various electrical components and subsystems of force sensing module 230.

Microprocessor 231 may be configured to receive data from one or more of force sensors 233a-233d and inertial measurement unit 221, and transmit the received data to one or more remote receivers. Accordingly, microprocessor 231 may include (or otherwise be coupled to) a wireless transceiver chipset, and may be configured communicate (e.g., wirelessly (as shown in FIG. 5, or using a wireline protocol) with, for example, processing system 310. As such, microprocessor 231 may be configured to transmit the detected force and orientation data received from one or more of sensors 233a-233d and inertial measurement unit 231 to processing system 310, for further analysis. As illustrated in FIG. 5, microprocessor 231 may be powered by power supply 232, such as a battery, fuel cell, MEMs micro-generator, or any other suitable compact power supply.

Force sensing module 230 may optionally include an inertial measurement unit 221 to provide orientation (and/or position) information associated with force sensing module 230 relative to a reference orientation (and/or position). Inertial measurement unit 221 may include one or more subcomponents configured to detect and transmit information that can be used to derive an orientation of the inertial measurement unit 221 (and, by extension, any object rigidly affixed to inertial measurement unit 221, such as a femur of the patient). Inertial measurement unit 221 may embody a device capable of determining a 3-dimensional orientation associated with any body to which inertial measurement unit 221 is attached. As explained previously and in accordance with certain exemplary embodiments, inertial measurement unit 221 may include one or more of a gyroscope 413, an accelerometer 414, or a magnetometer 415.

As illustrated in FIG. 6A, force sensing module may include a plurality of force sensors 233a-233d, each configured to measure respective force acting on the sensor. The type and number of force sensors provided within head 216 of prosthetic component can vary depending upon the resolution and the desired amount of data. For example, if the design goal of force sensing module 230 is to simply detect the magnitude of force present at the femoro-pelvic interface, then one sensor could be used. If, however, the design goal of force sensing module 230 is to not only provide the magnitude of the forces present at the femoro-pelvic interface, but also estimate the location of the center of the applied force, then additional sensors (as few as two, but, preferably, at least three) should be used to provide a sufficient number of data points to allow for accurate planar triangulation of the location of the center of the detected force. Furthermore, in embodiments where the design goal of force sensing module 230 is to provide independent (and simultaneous) monitoring of forces (both magnitude and location of center of force) applied at all regions of the substantially hemispheroidal head 216, then force sensing module 230 should include as few as four force sensors, but, preferably, at least six force sensors.

As illustrated in FIG. 6A, force sensing module 230 may include a plurality of force sensors 233a-233d. According to one embodiment, sensors 233a-233d may be mechanically coupled to the underside of substantially convex articular surface 601 of the housing at one end and to a rigid surface on the other end, so as to measure the compressive forces acting on the substantially convex articular surface 601. Furthermore, the connection points of sensors 233a-233d along the underside of substantially convex articular surface 601 of the housing may be relatively evenly distributed so as to provide adequate force sensing capability across the entirety of the surface. Although FIG. 6A illustrates embodiment 600 of the force sensing module as containing four force sensors, it is contemplated that additional force sensors may be used, depending on the desired resolution of the force measurements, as more sensors distributed across articular surface 601 will result in more accurate force magnitude and location measurements.

FIGS. 6B and 6C illustrate alternate configurations of embodiments in which the force sensing module is disposed within head 216 of femoral prosthetic component 200. FIG. 6B, for example, illustrates an embodiment with the trial femoral prosthetic component configured to detect force across the entire spherical surface of head 216. To provide adequate structural support, head 216 is adapted with internal structural reinforcement members 611, 612 to provide a rigid surface to which electronic circuit board 610 and force sensors 233a-233h may be mounted, respectively. FIG. 6C is similar to the embodiment illustrated in FIG. 6B, but uses a different type of force sensor (planar force sensor). For example the planar force sensor could be the FlexiForce® Load/Force Sensors from Tekscan, South Boston, Mass.

As shown in FIGS. 6B and 6C, the configurations of force sensing module 230 may be modified slightly to support a variety of different resistive or capacitive transducers for detecting applied force and/or pressure. Force sensors 233a-233h in FIGS. 6A and 6B each comprise two primary components: a metric portion that has a prescribed mechanical force-to-deflection characteristic and a measuring portion for accurately measuring the deflection of the metric portion and converting this measurement into an electrical output signal (using, for example, strain gauges). Some types of sensors include additional and/or different components than those listed above. For example, planar force sensors may include a flexible/compressible dielectric material (e.g., a polymer) and, rather than directly measuring the deflection of the material to determine the force, a change in force is determined based on change in an electrical parameter (e.g., capacitance) caused by the change in thickness of the compressed material. FIGS. 6A and 6B employ cantilever beam-type metric portion in accordance with certain exemplary disclosed embodiments. As shown by FIG. 6C, however, the designs of compartment 602 can be modified to support any of a variety of different configurations and type of transducers. Those skilled in the art will appreciate that transducers with additional or different mechanical deformation and sensing principles may be used without departing for the scope of the present disclosure. Indeed force sensors 233a-233h in FIGS. 6A and 6B may embody at least one type of the following configurations of force sensors: binocular, ring, shear, cantilever beam, or direct stress or spring torsion (including helical, disc, etc.) Alternatively or additionally, any suitable resistive sensor can be used as force sensors 233a-233h. In certain embodiments, the resistive strain gauge could be the transducer class S182K series strain gauges from Vishay Precision Group, Wendell N.C.

For embodiments 6A-6C, both the magnitude and locations of the load can be measured. Since embodiments 6A-6C may include an inertial measurement unit 221 for measurement of the orientation of the femoral ball relative to the acetabular cup, the location of the load can be measured relative to both the femoral ball and the acetabular cup articular surfaces. For example, since the femoral ball moves relative to the cup as the joint is articulated, the spatial location of the load on the femoral ball surface will change as the joint is moved regardless of a change in the load location in the cup. However, since the orientation of the ball may be independently measured in 3-dimensions by an inertial measurement unit, this change in load location on the femoral ball (in the absence of a change in the load location in the cup) is proportional to the magnitude and direction of the change in orientation and can be calculated from the orientation data and femoral component dimensions. In the presence of a change in the load location in the cup concurrent with a change in joint angle, the position of the load on the ball will be a summation of the two positional changes. Since the position change due to orientation change only can be calculated, it can be subtracted from the total positional change to calculate the change in load position in the cup alone.

FIGS. 7A and 7B provide respective cross-sectional side and overhead views of exemplary embodiments in which force sensing module 230 is embedded within a trial acetabular component 220 of trial hip prosthesis 200. Such embodiments may be particularly useful in measuring the forces applied to acetabular component of hip joint 110. According to the embodiments illustrated in FIGS. 7A and 7B, force sensing module 230 may include a housing 701 having a substantially concave articular surface 702 and an implant surface 703. According to one embodiment, the substantially concave articular surface 702 of housing 701 may be substantially hemispheroidal in shape. Housing 701 includes a compartment 704 defined by concave articular surface 702 and the implant surface 703. The substantially concave articular surface 702 (as well as the housing as a whole) may be made of a material with known (or calibrated) structural integrity, such that forces acting on the substantially concave articular surface 702 are transferred to force sensors (such as force sensors 233a-233d) disposed within compartment 704.

In the embodiment shown in FIG. 7A, force sensing module 230 may include an electronic circuit board 710, a microprocessor 231 (and corresponding power supply (not shown)), a first set of force sensors 233a-233b, a second set of force sensors 233d, 233e, and inertial measurement unit 221, each of which may be disposed within compartment 704. It is contemplated that additional and/or different components than those shown in FIG. 7A may be provided without departing from the scope of the present disclosure. For example, inertial measurement unit 221 may be excluded from force sensing module 230 altogether, or may be provided as an external component, affixed to the pelvis or some other portion of the acetabular prosthetic component 220 in addition to an inertial measurement unit on the femoral component.

Electronic circuit board 710 may include or embody any suitable material on which electronic circuits, such as processor 231, power supply (not shown), and inertial measurement unit 221 may be electrically coupled. For example, electronic circuit board 710 may embody a formed printed circuit board (PCB), multi-chip module (MCM), or flex circuit board. Electronic circuit board 710 may be configured to provide both integrated, space-efficient electronic packaging and mechanical support for the various electrical components and subsystems of force sensing module 230. According to the embodiment illustrated in FIG. 7A, electronic circuit board 710 may embody a flexible or form-fitted material that can be shaped or formed to conform to the shape of substantially hemispheroidal trial acetabular prosthetic component 220.

Microprocessor 231 may be configured to receive data from one or more of force sensors 233a-233d and inertial measurement unit 221, and transmit the received data to one or more remote receivers. Accordingly, microprocessor 231 may include (or otherwise be coupled to) a wireless transceiver chipset, and may be configured to communicate (e.g., wirelessly (as shown in FIG. 5, or using a wireline protocol) with, for example, processing system 310. As such, microprocessor 231 may be configured to transmit the detected force and orientation data received from one or more of sensors 233a-233d and inertial measurement unit 231 to processing system 310, for further analysis. As illustrated in FIG. 5, microprocessor 231 may be powered by power supply 232, such as a battery, fuel cell, MEMs micro-generator, or any other suitable compact power supply.

Force sensing module 230 may optionally include an inertial measurement unit 221 to provide orientation (and/or position) information associated with force sensing module 230 relative to a reference orientation (and/or position). Inertial measurement unit 221 may include one or more subcomponents configured to detect and transmit information that can be used to derive an orientation of the inertial measurement unit 221 (and, by extension, any object rigidly affixed to inertial measurement unit 221, such as the pelvis of a patient). Inertial measurement unit 221 may embody a device capable of determining a 3-dimensional orientation associated with any body to which inertial measurement unit 221 is attached. As explained previously and in accordance with certain exemplary embodiments, inertial measurement unit 221 may include one or more of a gyroscope 413, an accelerometer 414, or a magnetometer 415.

As illustrated in FIG. 7A, force sensing module may include a plurality of force sensors 233a-233d, each configured to measure respective force acting on the sensor. The type and number of force sensors provided within compartment 704 of trial acetabular prosthetic component 220 can vary depending upon the resolution and the desired amount of data. For example, if the design goal of force sensing module 230 is to simply detect the magnitude of force present at the femoro-pelvic interface, then one sensor could be used. If, however, the design goal of force sensing module 230 is to not only provide the magnitude of the forces present at the femoro-pelvic interface, but also estimate the location of the center of the applied force, then additional sensors (as few as two, but, preferably, at least three) should be used to provide a sufficient number of data points to allow for accurate planar triangulation of the location of the center of the detected force. Furthermore, in embodiments where the design goal of force sensing module 230 is to provide independent (and simultaneous) monitoring of forces (both magnitude and location of center of force) applied at all regions of the substantially hemispheroidal trial acetabular prosthetic component 220, then force sensing module 230 should include as few as four force sensors, but, preferably, at least six force sensors.

As illustrated in FIG. 7A, force sensing module 230 may include a plurality of force sensors 233a-233d. According to one embodiment, sensors 233a-233d may be mechanically coupled to the underside of substantially concave articular surface 702 of the housing 701 at one end and to a rigid surface on the other end, so as to measure the compressive forces acting on the substantially concave articular surface 702. Furthermore, the connection points of sensors 233a-233d along the underside of substantially concave articular surface 702 of the housing may be relatively evenly distributed so as to provide adequate force sensing capability across the entirety of the surface. Although FIG. 7A illustrates an embodiment of the force sensing module as containing four force sensors, it is contemplated that additional force sensors may be used, depending on the desired resolution of the force measurements, as more sensors distributed across articular surface 702 will result in more accurate force magnitude and location measurements.

FIG. 7B provides an overhead view of a trial acetabular prosthetic component 220 showing an exemplary layout of force sensors 233a-233f and electronic circuit board 710. In the embodiment illustrated in FIG. 7B, force sensors 233a-233f are configuration with cantilever type metric components that are commonly affixed at one end to a pedestal located at the vertex (or center) of the substantially hemispheroidal trial acetabular prosthetic component 220. The other ends of the cantilever beam are connected to a particular location associated with substantially concave articular surface 702. Cantilevers of force sensors 233a-233f may be different sizes in order to create a desired coverage pattern. For example, as illustrated in FIG. 7B, a first set of force sensors 233a-233c may be configured to couple proximate an edge area (705 of FIG. 7A) of trial acetabular prosthetic component 220, in order to provide force sensing capability at the periphery of the component. Since three force sensors are used in the embodiment of FIG. 7B, they may be distributed at approximately 120° apart. Importantly, first set of sensors may comprise more than three sensors, and may be spaced at different intervals, depending upon the desired force coverage profile of the particular application.

Similarly, a second set of sensors 233d-233f may be configured to couple proximate an interior area (706 of FIG. 7A) of trial acetabular prosthetic component 220, in order to provide force sensing capability at the interior of the component. Since three force sensors are used in the embodiment of FIG. 7B, they may be distributed at approximately 120° apart. Importantly, first set of sensors may comprise more than three sensors, and may be spaced at different intervals, depending upon the desired force coverage profile of the particular application.

As noted above with respect to FIGS. 6A-6C, the configurations of force sensing module 230 may be modified slightly to support a variety of different resistive or capacitive transducers for detecting applied force and/or pressure. Although FIGS. 7A and 7B are illustrated as cantilever-type force sensing modules, those skilled in the art will appreciate that transducers with additional or different mechanical deformation and sensing principles may be used without departing for the scope of the present disclosure. Further, as illustrated in FIGS. 6B and 6C, the designs of compartment 702 can be modified in a similar manner to support any of a variety of different configurations of transducers. Indeed force sensors 233a-233h may embody at least one type of the following configurations of force sensors: binocular, ring, shear, cantilever beam, or direct stress or spring torsion (including helical, disc, etc.) Alternatively or additionally, any suitable piezoresistive sensor can be used as force sensors 233a-233h.

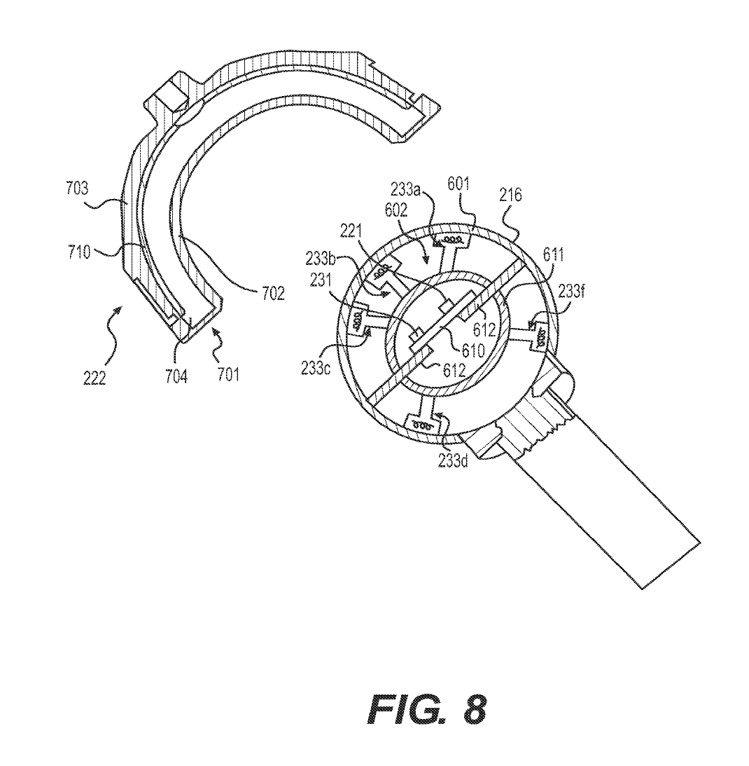

FIG. 8 illustrates an alternate embodiment for estimating the forces present at the femoro-pelvic interface. Rather than directly measuring the force using mechanical principles (as in FIGS. 6A-6C and 7A-7B), may be configured to measure force using proximity detection principles. As illustrated in FIG. 8, proximity sensors 233a-233e may be adapted as a plurality of inductive coils distributed around the interior of compartment 602 of head 216 of femoral component 200. Acetabular component 220 may be adapted to include a metallic sheet 710 within compartment 704. Compartment 704 and surface 702 are designed such that the distance between the two surfaces changes in response to changes in the loads at the interface. This can be achieved in a variety of ways including incorporation of compressible materials or springs between 710 and 702 and designing the housing 701 such that the surface 702 can move a small but sufficient distance with respect to 710. Alternatively, surface 702 can be constructed of a sufficiently flexible material such as polymer or any other material that by virtue of its material property and/or thickness allows sufficient movement of the surface 702 towards 710. The inductive proximity coils are configured to emit an oscillating field, which creates a high frequency electromagnetic field that emanates from substantially convex articular surface 601. When the inductive proximity coils are brought into proximity with a metallic component (such as metallic sheet 710 within trial acetabular prosthetic component 220), eddy currents are induced into the object. As the coils move closer to the metallic material (when, for example, head 216 compresses substantially concave articular surface 702 of acetabular component 220), eddy currents increase and result in an absorption of energy from the coil which dampens the oscillator amplitude. This dampening can be electrically measured and is proportional to the distance between the coil and metal object, which is indicative of a force value. Alternate electrical detection techniques that more directly measure a change in inductance may also be utilized.

Processes and methods consistent with the disclosed embodiments provide a system for monitoring the forces present at an orthopedic joint 110, and can be particularly useful in intra-operatively evaluating the performance of a reconstructed joint. As explained, while various components, such as force sensing module 230 and inertial measurement unit 221 can monitor various physical parameters (e.g., magnitude and location of force, orientation, etc.) associated with the bones and interfaces that make up orthopedic joint 110, processing system 310 provides a centralized platform for collecting and compiling the various physical parameters monitored by the individual sensing units of the system, analyzing the collected data, and presenting the collected data in a meaningful way to the surgeon. FIGS. 9, 10, and 11 illustrate exemplary processes and features associated with how processing system 310 performs the data analysis and presentation functions associated with orthopedic performance monitoring system 300.

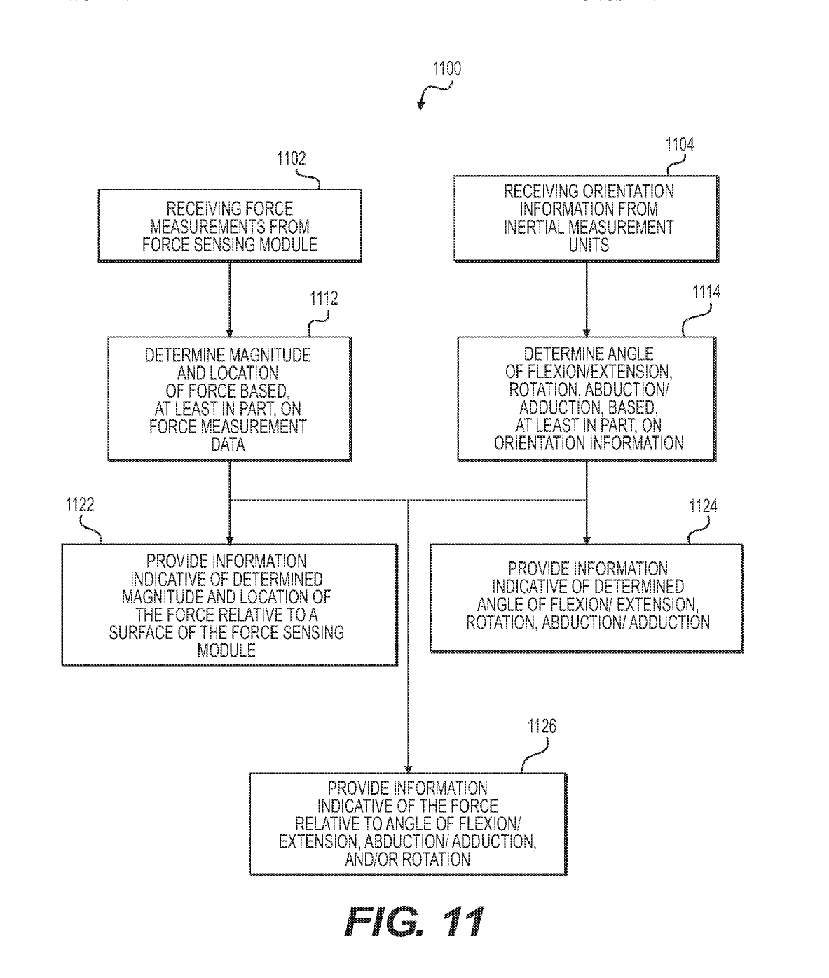

FIGS. 9 and 10 provide exemplary screen shots 900, 1000, respectively, corresponding to a graphical user interface (GUI) associated with processing system 310. Screen shots 900 may correspond to embodiments in which force sensing module 230 is configured to detect load distribution relative to the surface of the acetabular component 220. Screen shot 1000 may correspond to embodiments in which forces are tracked relative to the joint angles. Specific details for each of these screen shots will be described in detail below with respect to the exemplary processes and methods performed by processing system 310, as outlined in FIG. 11.

FIG. 11 provides a flowchart illustrating an exemplary data analysis process 1100 performed by processing system 310. As explained, processing system 310 may include software configured to receive, process, and deliver various performance data to other subcomponents and users associated with orthopedic performance monitoring system 300.

As illustrated in FIG. 11, the process may commence when processing system 310 receives force measurement information from force sensing module 230 (Step 1102) and/or orientation information from inertial measurement unit(s) 221 (Step 1104). As explained, the measurement information from force sensing module 230 may embody raw force data from each of force sensors, such as 233a-233h. Alternatively or additionally, the measurement information may include processed data indicative of a location and magnitude of a center of force applied at the femoro-pelvic interface. Orientation information from inertial measurement unit(s) 221 may include raw orientation sensor reading from one or more of gyroscope 413, accelerometer 414, or magnetometer 415. Alternatively or additionally, orientation information may include processed data indicative of the relative position of inertial measurement unit(s) 221 relative to a reference.

As explained, processing system 310 may include one or more communication modules for wirelessly communicating data with force sensing module 230 and/or inertial measurement unit(s) 221. As such, processing system 310 may be configured to establish a continuous communication channel with force sensing module 230 and/or inertial measurement unit(s) 221 and automatically receive force/performance and orientation/position data across the channel. Alternatively or additionally, processing system 310 may send periodic requests to one or more of force sensing module 230 and/or inertial measurement unit(s) 221 and receive updated performance parameters in response to the requests. In either case, processing system 310 receives force and orientation information in real-time or near real-time.

Processing system 310 may be configured to determine a magnitude and/or location of the center of the force detected by force sensing module 230 (Step 1112). In certain embodiments, force sensing module 230 may be configured to determine the location of the center of the force relative to the boundaries of the articular surface. In such embodiments, processing system 310 may not necessarily need to determine the location, since the determination was made by force sensing module 230.

In other embodiments, processing system 310 simply receives raw force information (i.e., a point-force value) from each sensor of force sensing module 230, along with data identifying which force sensor detected the particular force information. In such embodiments, processing system 310 may be configured to determine the location of the center of the force, by triangulating the center based on the relative value of a magnitude and the position of the force sensor within the force sensing module 230. Such triangulation algorithms are not disclosed in detail here, as such triangulation techniques are fairly well understood in the art

Processing system 310 may also be configured to determine an angle of flexion/extension, the angle of abduction/adduction, and/or the angle of internal/external rotation of joint 120 based on the orientation information received from inertial measurement unit(s) 221 (Step 1114). For example, processing system 150 may be configured to receive pre-processed and error-corrected orientation information from the inertial measurement unit(s) 140a, 140b. Alternatively, processing system 150 may be configured to receive raw data from one or more of gyroscope 243, accelerometer 244, and/or magnetometer 245 and derive the orientation based on the received information using known processes for determining orientation based on rotation rate data from gyroscope, acceleration information from accelerometer, and magnetic field information from magnetometer. In order to enhance precision of the orientation information, data from multiple units may be used to correct data from any one of the units. For example, accelerometer and/or magnetometer data may be used to correct error in rotation rate information due to gyroscope bias and drift issues. Optional temperature sensor information may also be utilized to correct for temperature effects.

Once processing system 310 has determined the magnitude and location of the center of the force detected by the force sensors and joint angles, processing system 310 may analyze and compile the data for presentation in various formats that may be useful to a user of orthopedic performance monitoring system 300. For example, as shown in FIG. 10, processing system 310 may be configured to display load distribution information relative to the center or boundaries of the acetabular prosthetic component 220 relative to the joint angles (flexion/extension, abduction/adduction, and internal/external). As part of a tracking feature, processing system 310 may be configured to compile data and display various representations of the compiled data. According to one embodiment, software associated with processing system 310 may analyze the compiled data and generate a graph 920 indicating the load distribution via color coding (e.g. center—green dot, center-edge—yellow dot, edge—red dot) at various joint angles over one or more cycles of articulations of the joint over it's full or partial range of motion.

In addition to magnitude values, processing system 310 may include a user interface element configured to display the instantaneous location of the center of the forces relative to the center or boundaries of the articular surface (Step 1122). In addition to the location, the graphical element may also be configured to adjust the size of the cursor or icon used to convey the location information to indicate the relative magnitude of the force value. For example, as illustrated in FIG. 10, processing system 310 may provide a user interface element 1020 that tracks, among other things, the location and magnitude of the center of the force relative to the acetabular cup center (see, e.g., UI element 220a).

For example, as an alternative or in addition to the magnitude and force presentation described above with respect to user interface regions 910, 920, processing system 310 may include user interface elements 1010, 1020 that provides information indicative of the instantaneous values for abduction and flexion, respectively, each of which processing system 310 can determine based on the orientation information from inertial measurement unit(s) 221 (Step 1124). Alternatively or additionally, processing system 230 may generate similar user interface elements (not shown) that depict the instantaneous values for flexion/extension and internal/external rotation. As part of this display element, processing system 310 may also display graphical representations of femur 140, pelvis 120, and force sensing module 230, based on the instantaneous position data received from inertial measurement unit(s) 221.

According to an exemplary embodiment, processing system 150 may also be configured to generate a user interface element that displays data that tracks the magnitude of force values as a function of flexion/extension angle, abduction/adduction angle, and internal/external rotation (Step 1026). For example, force magnitude information may be included with user interface elements 1010 and 1020, both of which display exemplary intra-operative orientation information (e.g., abduction/adduction and rotation).

It will be apparent to those skilled in the art that various modifications and variations can be made to the disclosed systems and methods for measuring orthopedic parameters associated with a reconstructed joint in orthopedic arthroplastic procedures. Other embodiments of the present disclosure will be apparent to those skilled in the art from consideration of the specification and practice of the present disclosure. It is intended that the specification and examples be considered as exemplary only, with a true scope of the present disclosure being indicated by the following claims and their equivalents.

What is claimed is:

1. A force sensing module for measuring performance parameters associated with an orthopedic articular joint, comprising:
a housing including a substantially concave articular surface and an implant surface, the substantially concave articular surface and the implant surface defining a compartment therebetween;
a first set of sensors disposed within the compartment, the first set of sensors being mechanically coupled between the substantially concave articular surface and the implant surface, the first set of sensors configured to detect information indicative of a first portion of a force present at a first area of the substantially concave articular surface; and
a second set of sensors disposed within the compartment, the second set of sensors being mechanically coupled between the substantially concave articular surface and the implant surface, the second set of sensors configured to detect information indicative of a second portion of the force present at a second area of the substantially concave articular surface, wherein the orthopedic articular joint is an articular joint of an upper extremity of a patient.

2. The force sensing module of claim 1, wherein the housing includes at least a portion of an acetabular cup or acetabular cup insert, the articular surface having a substantially hemispheroidal geometry configured to articulate with a corresponding portion of a prosthetic head.

3. The force sensing module of claim 1, wherein the first area includes an edge portion of the substantially concave articular surface and the second area includes an interior portion of the substantially concave articular surface.

4. The force sensing module of claim 1, wherein the first set of sensors includes at least three transducers, each transducer configured to detect a respective force value associated with the first portion of the force present at the first area of the substantially concave articular surface.

5. The force sensing module of claim 4, further configured to estimate, based at least in part on the force values detected by the first set of sensors, a magnitude and a location of a center of force associated with the first portion of the force present at the first area of the substantially concave articular surface.

6. The force sensing module of claim 1, wherein the second set of sensors includes at least three transducers, each transducer configured to detect a respective force value associated with the second portion of the force present at the second area of the substantially concave articular surface.

7. The force sensing module of claim 6, further configured to estimate, based at least in part on the force values detected by the second set of sensors, a magnitude and a location of a center of force associated with the second portion of the force present at the second area of the substantially concave articular surface.

8. The force sensing module of claim 1, further comprising a wireless transmitter disposed within the compartment and configured to wirelessly transmit the information indicative of the first and second portions of the forces to a remote processing module.

9. The force sensing module of claim 8, further comprising at least one inertial measurement unit disposed within the compartment and configured to detect information indicative of an orientation of the force sensing module relative to a reference.

10. The force sensing module of claim 9, wherein the at least one inertial measurement unit includes at least one of a gyroscope, an accelerometer, or a magnetometer.

11. The force sensing module of claim 9, wherein the at least one inertial measurement unit includes a gyroscope and an accelerometer.

12. The force sensing module of claim 1, further comprising a processor disposed with the compartment and coupled to the first and second sets of sensors, the processor configured to:
receive the information indicative of the first and second portions of the forces present at the respective first and second areas of the substantially concave articular surface; and
estimate a location of a center of the force relative to the substantially concave articular surface based, at least in part, on the received information indicative of the first and second portions of the forces present at the respective first and second areas of the substantially concave articular surface.

13. A computer-implemented method for tracking performance parameters associated with an orthopedic articular joint, the method comprising:
receiving, at a processor associated with a computer, first information indicative of a force detected at an articular surface of a prosthetic component of a patient;
estimating, by the processor, a location of a center of the force relative to the articular surface of the prosthetic component, the estimated location based, at least in part, on the first information; and
providing, by the processor, second information indicative of the estimated location of the center of the force relative to the approximate center of the articular surface of the prosthetic component, wherein the orthopedic articular joint is an articular joint of an upper extremity of a patient.

14. The computer-implemented method of claim 13, further comprising:
receiving, at the processor, third information indicative of an orientation of an anatomy of the patient relative to a reference position;
estimating, by the processor, at least one of an abduction/adduction angle or a flexion/extension angle associated with orthopedic articular joint, the at least one of the abduction/adduction angle or the flexion/extension angle, based, at least in part, on the third information; and
wherein the second information further includes information indicative of the at least one of the abduction/adduction angle or the flexion/extension angle associated with orthopedic articular joint.

15. The method of claim 14, wherein providing second information includes causing display of information indicative of the estimated location of the center of the force relative to the approximate center of the articular surface of the prosthetic component as a function of the at least one of the abduction/adduction angle or the flexion/extension angle associated with orthopedic articular joint.

16. The method of claim 13, further comprising:
estimating, by the processor, a magnitude and the location of the center of the force detected at the articular surface, the magnitude based, at least in part, on the first information;
wherein the second information is further indicative of a magnitude of the force detected at the articular surface.

17. The method of claim 16, wherein providing second information includes causing display of information indicative of the estimated location and magnitude of the center of the force relative to the approximate center of the articular surface of the prosthetic component.

18. The method of claim 13, wherein estimating the location of the center of the force relative the articular surface includes estimating a distance of the center of the force from a predetermined point on the articular surface.

19. The method of claim 18, wherein the predetermined point is a designated vertex of the articular surface.
